(19) United States
(12) Patent Application Publication
(10) Pub. No.: US 2026/0270703 A1
(43) Pub. Date: Sep. 10, 2026

(54) AN INTRAORAL SCANNING DEVICE CONFIGURED TO AUTHENTICATE MODE REQUEST

(71) Applicant: 3SHAPE A/S, Copenhagen K (DK)

(72) Inventor: Anders Robert JELLINGGAARD, Copenhagen K (DK)

(73) Assignee: 3SHAPE A/S, Copenhagen K (DK)

(21) Appl. No.: 18/875,411

(22) PCT Filed: Jun. 16, 2023
(86) PCT No.: PCT/EP2023/066211

§ 371 Date: Dec. 16, 2024

(30) Foreign Application Priority Data
Jun. 17, 2022 (EP) 22179517.2

(51) Int. Cl. H04W 12/069; A61C 9/00; G06F 21/33; G06F 21/44; H04W 12/30

(52) U.S. Cl. CPC H04W 12/069; A61C 9/0053; G06F 21/33; G06F 21/44; H04W 12/35

(57) ABSTRACT

A method and a handheld intraoral scanning device are disclosed. The handheld intraoral scanning device is configured to acquire intraoral scan data from a three-dimensional dental object during a scanning session. The handheld intraoral scanning device includes a processing unit configured to process intraoral scan data of a patient and provide 2D image data and/or 3D image data, a memory, and a wireless interface configured to transmit the 2D image data and/or the 3D image data, wherein the processing unit is configured to receive a mode request via the wireless interface, authenticate the mode request; and place the handheld intraoral scanning device into the requested mode if authentication of the mode request succeeds.

### FIELD

The present disclosure relates to an intraoral scanning device and in particular to intraoral scanning device and related method for configuration or operation of an intraoral scanning device.

### BACKGROUND

The functionality of an intraoral scanning device becomes increasingly advanced. Wireless communication between an intraoral scanning device and external devices, such as a clinic computer, a scan computer, a dental software on a computer, and a customization computer, has evolved. Typically, a wireless communication interface of an intraoral scanning device uses open standard-based interface. However, this poses many challenges in terms of security. An intraoral scanning device may assume any incoming data as legitimate, and may allow memory to be written or changed by an unauthorized party. Any such attacks may result in a malfunction of the intraoral scanning device, or a battery exhaustion attack.

However, an intraoral scanning device is a small device with strict constraints in terms of computational power, memory space, etc. Therefore, a device communicating with an intraoral scanning device cannot use an off-the-shelf security algorithm and protocol, at the risk of e.g. depleting the intraoral scanning device battery or degrading functions of the intraoral scanning device rendering the intraoral scanning quasi-useless.

Present intraoral scanning devices are part of a service infrastructure which includes communication between intraoral scanning devices, scan software for a specific service, and the provider of the service. The service could for example include manufacture of an aligner, a retainer, a crown, an implant, a bracer, a nightguard etc. For improving the usability of such an infrastructure for the dentist, minimal interaction between the infrastructure and the dentist is needed. One way of achieving this is by applying wireless communication between the intraoral scanning device and an external computer that is connected to a server that can forward the intraoral scan data to a service provider. Scan data of a patient can be characterized as being personal information, and therefore, there is a need for minimizing any risk of a third party stealing or corrupting the at least scan data. The scan data is characterized as personal information, and in some situations, other type of personal information is associated with the scan data, such as age, gender, location address, personal security number etc. In this example, a demand for improving the security of the wireless communication in the service infrastructure is needed.

### SUMMARY

An aspect of the present disclosure is to reduce risk of a third party accessing any part of the intraoral scanning device. There is a need for an intraoral scanning device that is protected against unauthorized modification of the intraoral scanning device and operation thereof.

A further aspect of the present disclosure is to provide an intraoral scanning device, and a method which seeks to mitigate, alleviate, or eliminate a third party's possibility to steal and/or corrupt personal information of the patient.

Yet another aspect of the present disclosure is to improve security of an intraoral scanning device. Security comprises in assessing threats, vulnerabilities and attacks and developing appropriate safeguards and countermeasures to protect against threats and attacks. The present disclosure relates to an intraoral scanning device comprising a processing unit configured to process intraoral scan data of a patient and provide 2D image data and/or 3D image data.

According to the aspect, a handheld intraoral scanning device for acquiring intraoral scan data from a three-dimensional dental object during a scanning session is disclosed. The handheld intraoral scanning device may comprise a processing unit configured to process intraoral scan data of a patient and provide 3D image data; a memory; and a wireless interface configured to transmit the 3D image data, wherein the processing unit is configured to receive a mode request via the wireless interface when no 3D image data is being transmitted, wherein the mode request is one or more of a service mode request for a service mode, a customization mode request for customizing a user interface of the handheld intraoral scanning device, an upgrade mode for upgrading the handheld intraoral scanning device and a debug mode request, wherein the service mode is characterized in that a firmware part of the memory is writable; authenticate the mode request to confirm that the mode request is valid for the handheld intraoral scanning device; and place the handheld intraoral scanning device into the requested mode if authentication of the mode request succeeds.

According to the aspect, a method for configuration of a haneheld intraoral scanning device that may comprise a processing unit configured to process intraoral scan data of a patient and provide 3D image data is discloses. The handheld intraoral scanning device may further include a memory unit and a wireless interface configured to transmit the 3D image. The method may comprise receiving a mode request via the wireless interface when no 3D image data is transmitted, wherein the mode request may be one or more of a service mode request for updating firmware data, a customization mode request, an upgrade mode request and a debug mode request, and wherein the service mode implies that a firmware part of the memory is writable. Furthermore, the method may comprise authenticating the mode request to confirm that the mode request is valid for the handheld intraoral scanning device, and placing the intraoral scanning device into the requested mode if authentication of the mode request succeeds.

According to the aspect, an intraoral scanning device for acquiring intraoral scan data from a three-dimensional dental object during a scanning session is disclosed. The intraoral scanning device may comprise a processing unit configured to process intraoral scan data of a patient; a memory; and a wireless interface configured to transmit the intraoral scan data of the patient, wherein the processing unit is configured to receive a mode request via the wireless interface, wherein the mode request is one or more of a service mode request for a service mode, a customization mode request, an upgrade mode and a debug mode request, wherein the service mode is characterized in that a firmware part of the memory is writable; authenticate the mode request; and place the intraoral scanning device into the requested mode if authentication of the mode request succeeds.

According to the aspect, an intraoral scanning device for acquiring intraoral scan data from a three-dimensional dental object during a scanning session is disclosed. The intraoral scanning device may comprise a processing unit configured to process intraoral scan data of a patient; a memory; and a wireless interface configured to transmit the intraoral scan data of the patient, wherein the processing unit is configured to receive a mode request via the wireless interface, wherein the mode request is one or more of a service mode request for a service mode, a customization mode request, an upgrade mode and a debug mode request, wherein the service mode is characterized in that a part of the memory is writable; authenticate the mode request; and place the intraoral scanning device into the requested mode if authentication of the mode request succeeds.

According to the aspect, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data; a memory; and a wireless interface configured to transmit the 2D image data and/or the 3D image data, wherein the processing unit is configured to receive an instruction request via the wireless interface, wherein the instruction request is one or more of a service instruction request for a service instruction, a customization instruction request, an upgrade instruction request and a debug instruction request, wherein the service instruction is characterized in that a firmware part of the memory is writable; authenticate the instruction request; and place the intraoral scanning device into the requested instruction if authentication of the instruction request succeeds.

A mode request may be similar to an instruction request. For example, during transmission of data packages via the wireless communication link to the intraoral scanning device, each data package is being authenticated or verified based on a signature, and when all data packages are being successful authenticated or verified then the intraoral scanning mode is placed into a service instruction which results in installation of the data packages into the firmware part of the memory.

According to the aspect, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data; a memory; and a wireless interface configured to transmit the 2D image data and/or the 3D image data, wherein the processing unit is configured to receive a connection request via the wireless interface, wherein the connection request is one or more of a service connection request for a service connection, a customization connection request, an upgrade connection request and a debug connection request, wherein the service connection is characterized in that a firmware part of the memory is writable; authenticate the connection request; and place the intraoral scanning device into the requested connection if authentication of the connection request succeeds.

A mode request, an instruction request, a connection request, a memory request, or a state request may be similar but with few distinguishing elements in relation to how the intraoral scanning device is being configured to receive data packages from an external device and install the data packages.

The handheld intraoral scanning device may receive the mode request when no 3D image data is being transmitted via the wireless interface. The

The intraoral scanning device may be placed into a requested mode which configures the intraoral scanning device to perform changes to how the images are being acquired by the optical unit, and how the processing unit is processing the images into image data, such as 2D image and/or 3D image.

An intraoral scanning device is in a scanning session when it is being used intentionally, such as for scanning an oral cavity of a patient.

The intraoral scanning device may be a handheld scanning device for scanning inside an oral cavity of a patient. The intraoral scanning device differs from other type of teeth scanning devices in that the intraoral scanning device is a handheld scanning device which can easily be handled by one hand by a user, and which has no wired connection to any external device during scanning of an inside of an oral cavity of a patient. Therefore, the only attack which an intraoral scanning device may experience is via the wireless interface.

The intraoral scanning device refers to a device configured to conduct a scan inside the oral cavity of a patient, or a part thereof, or parts thereof, such as a tooth, teeth, gingiva, etc., or to obtain a 2D image data and/or 3D image data of the oral cavity of a patient or parts thereof, such as a tooth, teeth and/or gingiva, etc. the intraoral scanning device may be an intraoral scanner that is fully or partly inserted in the oral cavity of a patient, such as a wireless intraoral scanning device.

The method and the intraoral scanning device as disclosed provide secure configuration of the intraoral scanning device, such as secure access to the memory of the intraoral scanning device. It is an advantage of the present disclosure that the intraoral scanning device can only be configured or updated by authorized parties. The disclosed intraoral thus has the advantage of detecting and preventing any modification by unauthorized parties. The intraoral scanning device disclosed herein is advantageously protected against attacks such as spoofing attacks, man-in-the-middle attacks, and/or replay-attacks.

The intraoral scanning device is the key element in providing the needed level of security in wireless communication in a service infrastructure which at least includes the intraoral scanning device and a scan computer or a dental software on a computer. It would not be possible for a third party to attack the wireless communication as this person needs to have the intraoral scanning device physically in its hand. It would not even be enough to have access to the scan computer or the dental software.

The method as disclosed herein provides a secure configuration and/or update of an intraoral scanning device.

The present disclosure provides improved security of an intraoral scanning device. Security comprises assessing threats, vulnerabilities and attacks and developing appropriate safeguards and countermeasures to protect against threats and attacks.

The intraoral scanning device comprises a processing unit. The processing unit may be configured to process intraoral scan data of a patient and provide 2D image data and/or 3D image data. The 2D image data and/or 3D image data may include information about the anatomy of the oral cavity of the patient, such as teeth, gingival, bone level, and/or information about diagnostic indicators such as caries, bone loss, gingivitis, gingiva recession, periodontitis, bone loss, cracks, and occlusion.

The 2D image data and/or the 3D image data may be image data configured to be visualizable on a display in a 2D or a 3D manner, respectively.

The intraoral scanning device may be operated in one or more modes. The one or more modes may include a first mode and/or a second mode. The one or more modes may include a third mode and/or a fourth mode. The one or more modes may include a default mode.

The first mode may be a service mode. A service mode may be characterized in that a firmware part of the memory can be written in the service mode. The firmware part of the memory may be write-protected in at least one other mode of the intraoral scanning device. Furthermore, the service mode may include setting the intraoral scanning device in a state where the optical unit of the intraoral scanning device is preparing to be used, for example, by heating up the light projector(s) and/or turning on the image sensor. Furthermore, the service mode may include setting the intraoral scanning device in a state where the intraoral scanning device is performing a self-check of moving parts, such as a moveable focus lens, an intensity of the light projector(s) and/or signal-to-noise of the image sensor. Other elements of the intraoral scanning device could be susceptible for a self-check but is not mentioned in this disclosure.

The second mode may be a customization mode. A customization mode may be characterized in that a customization part of the memory can be read and/or written in the customization mode. A customization mode may be characterized in that a firmware part of the memory is write-protected. The customization part of the memory may comprise setting data, such as power management settings, configuration of a user interface of the intraoral scanning device and/or settings of an optical unit of the intraoral scanning device. An intraoral scanning device may include a color image sensor, such as an RGB image sensor, and in the customization mode, different color areas may be configured to be deactivated and/or activated during at least a scanning session. Thus, the customization part of the memory may comprise data that relates to which color areas of the RBG image sensor should be activated or deactivated during a scanning session. An intraoral scanning device may include a monochromatic image sensor and colored light emitting diodes, and in the customization mode, the different colored light emitting diodes may be configured to be deactivated and/or activated during a scanning session. Thus, the customization part of the memory may comprise data that relates to which colored light emitting diodes should be activated or deactivated during a scanning session. A colored light emitting diode may be configured to emit light with a color, such as blue, red, green etc. In another example, the the intraoral scanning device could include one or more near-infrared light emitting diodes which also can be set to be activated and/or deactivated during a scanning session in the customization mode.

The optical unit may include one or more light projectors, one or more optical components, and one or more image sensors.

The user interface of the intraoral scanning device may include at least a touch sensor, at least a touch button, at least a light emitting diode, a haptic sensor, and/or an accelerometer. The handheld intraoral scanning device may include a motion sensor which is configured to sense the motion of the handheld intraoral scanning device. The handheld intraoral scanning device is configured to communicate wirelessly with an external device that is connected to a display. A cursor on the display may be moved around based on motion signals provided by the motion sensor to the external device. The user is able to navigate the cursor on the display by moving the handheld intraoral scanning device. The service mode request may include settings update that relates to the motion sensor of the handheld intraoral scanning device, and the customization mode request may relate to a customization of a user interface of the handheld intraoral scanning device which may involve a graphical setup of a graphical user interface on the display. For example, when the handheld intraoral scanning device connects to the external device, the handheld intraoral scanning device forwards a customization package to the external device via the wireless interface, and the external device is then configured to change the graphical setup based on the customization package. The customization package may be updated by the customization mode request.

The third mode may be a debug mode. A debug mode may be characterized in that a debug part of the memory can be read and/or written in the customization mode. A debug mode may be characterized in that a customization part of the memory can be read and/or written in the debug mode. A debug mode may be characterized in that a firmware part of the memory can be read and/or written in the debug mode. The debug part of the memory may be read-protected and/or write-protected in at least one other mode of the intraoral scanning device, such as in the default mode and/or the customization mode. In debug mode, the handheld intraoral scanning device may be configured to transmit debug data that relates to the performance of the handheld intraoral scanning device, such as a temperature within the handheld intraoral scanning device during a scanning, the performance of the light projector and the image sensor of the handheld intraoral scanning device. Furthermore, the debug data may relate to the performance of the wireless interface during scanning and when no scanning is being performed.

The firmware data may include updates to the handheld intraoral scanning device that improves the functionality and features of the device.

The fourth mode may be an upgrade mode. An upgrade mode may be characterized in that an upgrade part of memory can be read and/or written in the upgrade mode. An upgrade mode may be characterized in that a firmware part of the memory is write-protected. The upgrade part of the memory may comprise intraoral scanning device data, such as improved features, new features relating to an operating software system, a FPGA or other electronic/digital hardware of the intraoral scanning device, such as a scanner throttle, a focus lens motor, a light projector(s), and/or image sensor.

The default mode may be a boot mode. A boot mode may be characterized in that the intraoral scanning device may be operated according to operating parameters set during booting and/or in response to user input via the user interface. The user input may include entering a scan mode, stop the scan mode, entering a command mode where the intraoral scanning device functions as a pointer in a software application, i.e. when moving the scanner then the cursor/pointer in the software moves correspondingly. The default mode may be characterized in that the firmware part (or at least a part thereof) and/or the customization part of the memory (or at least a part thereof) is write-protected and/or read-protected in the default mode. The default mode may be characterized in that the debug part of the memory (or at least a part thereof) is read-protected and/or write-protected in the default mode.

The intraoral scanning device may comprise a memory. The memory may be embedded in the processing unit and/or be employed in a memory unit connected to the processing unit. The memory may comprise a first memory part. The first memory part may be a firmware part of the memory. The firmware part of the memory may be configured to be accessed in the service mode e.g. to be written to and/or read from in the service mode. The firmware part of the memory may additionally be configured to be accessed in the debug mode. The memory may comprise a second memory part. The second memory part may be a customization part of the memory. The customization part of the memory may be configured to be accessed in the customization mode e.g. to be written to and/or read from in the customization mode. The customization part of the memory may additionally be configured to be accessed in the service mode and/or the debug mode. The memory may comprise a third memory part. The third memory part may be a debug part of the memory. The debug part of the memory may be configured to be accessed in the debug mode e.g. to be written to or read from in the debug mode. The memory may comprise a fourth memory part, The fourth memory part may be an upgrade part of the memory. The upgrade part of the memory may be configured to be accessed in the upgrade mode, e.g. to be written to or read from in the upgrade mode.

The intraoral scanning device may comprise a wireless interface configured to enable wireless communication between the intraoral scanning device and another device. The wireless interface may comprise a wireless transceiver, e.g. configured for wireless communication at frequencies in the range from 2.4 to 2.5 GHz, 2.4 GHz to 5 GHz, about 2.45 GHz or about 5 GHz. The wireless transceiver may be a Bluetooth transceiver, a Bluetooth Low Energy transceiver, or a Wireless Fidelity (WIFI) transceiver. The wireless interface may form a connection to one or more other devices such as a computer, and/or a scan computer, and/or a tablet and/or a smart phone.

The processing unit/intraoral scanning device may be configured to receive a mode request via the wireless interface. The mode request may comprise a mode identifier indicative of the requested mode. The mode request may be a service mode request, e.g. the mode identifier is indicative of a first/service mode. The mode request may be a customization mode request, e.g. the mode identifier is indicative of a second/customization mode. The mode request may be a debug mode request, e.g. the mode identifier is indicative of a third/debug mode. The mode request may be an upgrade mode request, e.g. the mode identifier is indicative of a fourth/upgrade mode. Accordingly, the mode request may be one of a service mode request, a customization mode request, an upgrade mode request and a debug mode request.

The intraoral scanning device may be placed into the requested mode if the intraoral scanning device is not placed in a scanning session. The intraoral scanning device is scanning in a scanning session when being placed in the scanning session.

The placing of the intraoral scanning device into the requested mode may be scheduled for a specific time on a day when the intraoral scanning device will not be used. The scheduling may be determined by the processing unit based on historical usage time of the intraoral scanning device and a machine learning model. The machine learning model receives timestamps from a clock in the intraoral scanning device and input information about when the intraoral scanning device is being used in a scanning session. The machine learning model includes a training data set which includes historical usage time of the intraoral scanning device being in the scanning session. Based on the machine learning model and a timestamp defining the time of the day the processing unit will know when to be set into a requested mode if receiving a mode request. The advantage of the scheduling is that a valid authenticated mode request will not interfere the work of the dentist with the intraoral scanning device. Furthermore, when being placed into the customization mode, the intraoral scanning device can be programmed to do time consuming updates within specific time-period(s). For example, an update which last more than 30 mins will automatically be planned to be performed in a time-period of more than 30 mins where the intraoral scanning device will not be used, such as outside the working hours or during a break of the dentist/clinic.

The processing unit may be configured to place the intraoral scanning device into the requested mode if authentication of the mode request succeeds and if a timestamp is within a time-period. The timestamp is generated by a clock of the intraoral scanning device and received by the processing unit.

The processing unit may include a machine learning model that includes a training data set which includes historical data the relates to usage time of the intraoral scanning device being in a scanning session, and wherein the machine learning model receives a timestamp from a clock in the intraoral scanning device and input information about when the intraoral scanning device is being used in a scanning session, and the processing unit may then be configured to place the intraoral scanning device into the requested mode if authentication of the mode request succeeds and if the machine learning model outputs a trigger that allows the intraoral scanning device to be placed into the mode.

The mode request may comprise a sender identifier indicative of the mode request sender. The mode request may comprise a certificate, such as a digital signature, for certifying the mode request sender. This allows for direct authentication of the mode request. The mode request may comprise a session identifier, e.g. an encrypted session identifier.

The intraoral scanning device may be paired with a sender of the mode request prior to receipt of the mode request. In the pairing, the intraoral scanning device and the sending/client device may have exchanged one or more of intraoral scanning device identifier, sender identifier, session identifier, etc.

The processing unit/intraoral scanning device is configured to authenticate the mode request and to place the intraoral scanning device into the requested mode if authentication of the mode request succeeds. The processing unit may be configured to place the intraoral device into a mode different from the requested mode, such as the default mode, if authentication of the mode request fails.

The intraoral scanning device disclosed herein has the advantage of verifying integrity of received mode requests and/or senders thereof, detecting any alteration and disregard altered mode requested. The intraoral scanning device disclosed herein may advantageously allow access to specific parts of the memory only with authenticated parties, such as an authenticated scan computer, an authenticated computer, an authenticated accessory device, an authenticated external device and/or an authenticated server.

The processing unit may be configured to authenticate the mode request by authenticating the sender of the mode request.

The processing unit/intraoral scanning device may be configured to authenticate the mode request by verifying integrity of a digital signature of the mode request. The processing unit may be configured to authenticate the mode request by verifying integrity of the mode request. The mode request may comprise a message authentication code (MAC). To verify integrity of the mode request may comprise to verify the message authentication code, e.g. with a session identifier stored in the intraoral scanning device. The mode request may comprise a digital signature or certificate. To verify integrity of the mode request may comprise verifying the digital signature or certificate.

The processing unit/intraoral scanning device may be configured to send a mode response. For example, to place the intraoral scanning device into the requested mode if authentication of the mode request succeeds may comprise sending a mode response. The processing unit/intraoral scanning device may be configured to generate and/or send a mode response in response to the mode request. The processing unit may be configured to obtain and/or store a session identifier (may also be denoted session key) and include the 13rocessn identifier and/or an encrypted version thereof in the mode response. To obtain the session identifier may comprise to generate the session identifier, e.g. as a random or pseudo-random number. Thus, the intraoral scanning device and/or the processing unit may comprise a number generator, e.g. configured to generate a random or pseudo-random number as a session identifier. By using a unique session identifier or session identifier from a large number of available session identifiers, the processing power requirements in the intraoral scanning device may be reduced. Further, simple encryption is facilitated and replay-attacks are prevented.

The processing unit may be configured to encrypt the session identifier, optionally based on an intraoral scanning device key. The session identifier may be a session key in the form of a symmetric key. A symmetric session key may provide a lightweight processing of the security algorithms on the processing unit, such as lightweight encryption, lightweight decryption, lightweight integrity protection, etc. The intraoral scanning device key may be a symmetric key or a public key of a private-public key pair. The intraoral scanning device key may be stored in a permanent memory of the intraoral scanning device, e.g. during manufacture or during a customization session.

The mode response may comprise the encrypted session key. The session response may comprise an intraoral scanning device identifier and/or the session key. Thus, the processing unit may be configured to send an intraoral scanning device identifier and/or the session key in the mode response. A mode response comprising an intraoral scanning device identifier may enable the sender of the mode request to obtain the intraoral scanning device key, either from a database or by requesting the intraoral scanning device key from the manufacturer, which in turn enables the sender of the mode request to decrypt an encrypted session identifier/key and use the session identifier when sending data to the intraoral scanning device.

The mode request may be received in a session. The processing unit/intraoral scanning device may be configured to terminate the session if authentication of the mode request fails. The mode request may comprise a signature, and to authenticate the mode request may comprise to verify the signature of the mode request.

The processing unit may be configured to obtain, e.g. generate a session identifier, e.g. upon receipt of the mode request or when the intraoral scanning device is in a service mode, a customization mode, or a debug mode. The processing unit may be configured to encrypt the session identifier, e.g. with an intraoral scanning device key. The processing unit may be configured to transmit the session identifier or the encrypted session identifier via the wireless interface, e.g. as a part of the mode response or a session setup message. The processing unit may be configured to store the session identifier in the intraoral scanning device.

The processing unit may be configured to receive data via the wireless interface, e.g. when the intraoral scanning device is in a mode, e.g. the service mode, the customization mode and/or the debug mode. The processing unit may be configured to authenticate the received data, e.g. when the intraoral scanning device is in one or more modes, e.g. the service mode, the customization mode and/or the debug mode. The processing unit may be configured to store intraoral scanning device data in a part of the memory based on the received data if authentication of the data succeeds. For example, when the intraoral scanning device is in a service mode, the processing unit may store intraoral scan data, such as e.g. firmware, based on the received data in the firmware part of the memory. In an exemplary intraoral scanning device, the processing unit may, when the intraoral scanning device is in a customization mode, store intraoral scan data (such as customization data) based on the received data in the customization part of the memory. In an exemplary intraoral scanning device, the processing unit may, when the intraoral scanning device is in a debug mode, store intraoral scanning device data (debug data) based on the received data in the debug part of the memory.

The processing unit may be configured to authenticate the received data by verifying integrity of the received data. Verifying integrity of the received data may be based on the session identifier stored in the intraoral scanning device. The received data may comprise a message authentication code. To verify integrity of the received data may comprise to verify the message authentication code, e.g. with the stored session identifier. The received data may comprise a digital signature. To verify integrity of the received data may comprise verifying the digital signature.

The data may comprise a session identifier, and to authenticate the data may comprise to compare the session identifier of received data with the session identifier stored in the intraoral scanning device.

The data may be received in a session. The processing unit may be configured to terminate the session if authentication of the received data fails, e.g. the processing unit may be configured to terminate the session if integrity of the received data is corrupted, i.e. verification of the integrity fails. The processing unit may be configured to place the intraoral scanning device in another mode, such as the default mode, if authentication of the received data fails.

The intraoral scanning device/processing unit may be configured to receive a mode exit request and to place the intraoral scanning device in another mode, such as the default mode, e.g. if an authentication of the mode exit request succeeds. For example, a client device may send a mode exit request when customization or transfer of firmware is done.

The disclosed method provides secure configuration and/or update of an intraoral scanning device. The method may comprise placing the intraoral scanning device into a default mode if authentication of the mode request fails. The method may comprise determining if operation in default mode fails and switching to service mode if operating the intraoral scanning device in default mode fails.

In the method, authenticating the mode request may comprise authenticating the sender of the mode request.

In the method, the mode request may comprise a digital signature, and authenticating the mode request may comprise verifying the digital signature.

In the method, authenticating the mode request may comprise verifying integrity of the mode request.

The method may comprise receiving data via the wireless interface, e.g. when the intraoral scanning device is in one or more modes, e.g. the service mode, the customization mode, the upgrade mode and/or the debug mode. The method may comprise authenticating the received data, e.g. when the intraoral scanning device is in one or more modes, e.g. the service mode, the customization mode, the upgrade mode and/or the debug mode. The method may comprise storing intraoral scanning device data in a part of the memory based on the received data if authentication of the data succeeds. For example, when the intraoral scanning device is in a service mode, the method may comprise storing intraoral scanning device data (firmware) based on the received data in the firmware part of the memory. In an exemplary method, the method may, when the intraoral scanning device is in a customization mode, comprise storing intraoral scanning device data (such as customization data, scanning settings) based on the received data in the customization part of the memory. In an exemplary method, the method may, when the intraoral scanning device is in a debug mode, comprise storing intraoral scanning device data (debug data) based on the received data in the debug part of the memory. In an exemplary method, the method may, when the intraoral scanning device is in an upgrade mode, comprise storing intraoral scanning device data (such as data including improved features, new features relating to an operating software system, a FPGA or other electronic/digital hardware of the intraoral scanning device) based on the received data in the debug part of the memory. The method may comprise placing the intraoral scanning device in another mode, such as the default mode, if authenticating the received data fails.

The processing unit may be configured to operate the intraoral scanning device in default mode, and switch to service mode if operating the intraoral scanning device in default mode fails.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates an exemplary architecture according to this disclosure;

FIG. 2 illustrates an exemplary intraoral scanning device;

FIG. 3 shows an exemplary sequence diagram between an intraoral scanning device and a client device;

FIG. 4 shows an exemplary sequence diagram; and

FIG. 5 illustrates an exemplary flowchart of a method.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intraoral scan data may be performed by a dental scanning system that may include an intraoral scanning device such as the TRIOS series scanners from 3Shape A/S. The dental scanning system may include a wireless capability as provided by a wireless network unit. The intraoral scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the intraoral scanning device is operated by projecting a pattern and translating a focus plane along an optical axis of the intraoral scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the intraoral scanning device relative to the object or imaging the object at a different view, a new stack of 2D images for that view may be captured. The focus plane position may be varied by means of at least one focus element, e.g., a moving focus lens. The intraoral scanning device is generally moved and angled during a scanning session, such that at least some sets of sub-scans overlap at least partially, in order to enable stitching in the post-processing. The result of stitching is the digital 3D representation of a surface larger than that which can be captured by a single sub-scan, i.e. which is larger than the field of view of the 3D scanning device. Stitching, also known as registration, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of an intraoral scanning device is a triangulation scanner, where a time varying pattern is projected onto the dental object and a sequence of images of the different pattern configurations are acquired by one or more cameras located at an angle relative to the projector unit.

The intraoral scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono-or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the intraoral scanning device comprises a light source configured to excite fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The intraoral scanning device preferably further comprises optical components for directing the light from the light source to the surface of the dental object. The specific arrangement of the optical components depends on whether the intraoral scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the Illumination of the dental object is directed, using optical components of the intraoral scanning device, towards the image sensor(s). The image sensor(s) are configured to generate a plurality of images based on the incoming light received from the illuminated dental object. The image sensor may be a high-speed image sensor such as an image sensor configured to acquire images with exposures of less than 1/1000 second or frame rates in excess of 250 frames pr. Second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit. The wireless network unit is configured to wirelessly connect the dental scanning system to the network comprising the plurality of network elements including the at least one network element configured to receive the processed data.

The dental scanning system preferably further comprises a processor configured to generate scan data (such as intraoral scan data) by processing the two-dimensional (2D) images acquired by the intraoral scanning device. The processor may be part of the intraoral scanning device. As an example, the processor may comprise a Field-programmable gate array (FPGA) and/or an Advanced RISC Machines (ARM) processor located on the intraoral scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the intraoral scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The intraoral scanning device may be configured to transmit other types of data in addition to the scan data. Examples of data include 3D information, texture information such as infra-red (IR) images, fluorescence images, reflectance color images, x-ray images, and/or combinations thereof.

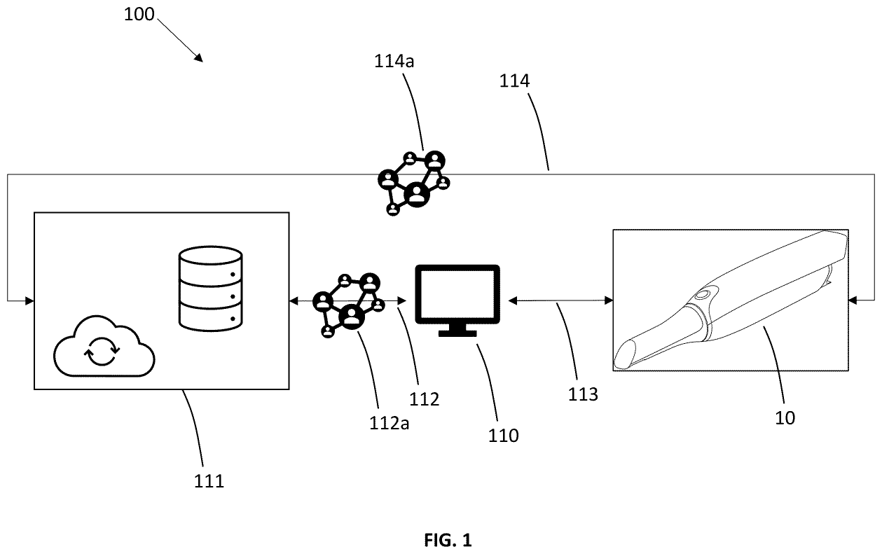

FIG. 1 illustrates an exemplary architecture 100 according to this disclosure. The architecture 100 comprises an intraoral scanning device 10, a client device 110, and a server device 111. The client device 110 may comprise a computing device acting as a client, a customization device, a handheld device, a relay, a tablet, a personal computer, a mobile phone, and/or USB dongle plugged into a personal computer. The server device 111 may comprise a computing device configured to act as a server, i.e. to serve requests from the client device 110 and/or from the intraoral scanning device 10. The server device 111 may be controlled by the intraoral scanning device manufacturer.

The intraoral scanning device 10 may be connected to the client device 110 via a communication link 113, such as a wireless communication link or a bidirectional wireless communication link. The wireless communication link may be carried over a short-range communication system, such as Bluetooth, Bluetooth low energy, IEEE 802.11, Zigbee, WIFI. The intraoral scanning device 10 may be connected to the client device 110 over a network.

The intraoral scanning device 10 may be connected to the server device 111 via a wireless communication link 114 or a bidirectional wireless communication link 114 over a network 114a, such as a bidirectional wireless communication link and/or wireless communication link over a network.

The client device 110 may be connected to the server device 111 via a communication link 112 over a network 112a, such as a bidirectional wireless communication link and/or wireless communication link over a network. In an embodiment, the network 112a may be the Internet.

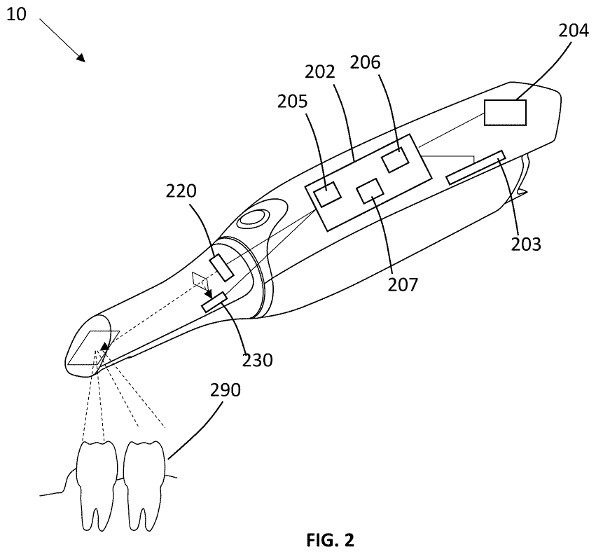

FIG. 2 illustrates an exemplary intraoral scanning device 10. The exemplary intraoral scanning device 10 comprises a processing unit 202 configured to process intraoral scan data of a patient 290 and provide 2D image data and/or 3D image data. The exemplary intraoral scanning device 10 comprises a memory and a wireless interface 204. The memory is in FIG. 2 illustrated in the form of a memory unit 203 external to the processing unit 202. The memory may in other exemplary intraoral scanning devices be at least partly embedded in the processing unit 202 and/or in the memory unit 203.

The processing unit 202 is configured to receive a mode request via the wireless interface 204. Hence, the processing unit 202 comprises a receive/send unit 205 configured to send and/or receive via the wireless interface 204. The receive/send unit 205 is configured to send and receive via the wireless interface 204 to/from an external device, such as a server device, a client device, a customization device, an accessory, a relay device, a smart phone. The processing unit 202 is configured to authenticate the mode request. Hence, the processing unit 202 may comprise an authenticator 206 configured to authenticate the mode request. The processing unit 202 is configured to place the intraoral scanning device into the requested mode, such as a service mode, a customization mode, an upgrade mode or debug mode, if authentication of the mode request succeeds. Hence the processing unit 202 comprises a mode controller 207 configured to place the intraoral scanning device 10 into the requested mode, e.g. based on an output from the authenticator 206. In the intraoral scanning device in FIG. 2, the processing unit 202 is configured to place the intraoral scanning device into a default mode if authentication of the mode request fails, the default mode comprising booting the intraoral scanning device and operating the intraoral scanning device according to operating parameters set during booting. In an embodiment, the operating parameters set during booting may be stored in a non-volatile part of the memory unit 203. In an embodiment, the operating parameters set during booting may comprise a default setting enabling the intraoral scanning device to function according to a default setting programmed during production of the intraoral scanning device.

The intraoral scanning device comprises a light projector 220 and an image sensor 230. The light projector includes at least one or more light emitting diodes and/or one or more infrared light source for emitting light pattern to a three-dimensional dental object 290 of a patient or of a wax model 290 which is a replicate of the patient's dental. The image sensor 230 receives the reflective light from the dental object 290, and the image sensor 230 converts the reflected light into intraoral scan data. The processing unit 202 is then configured to process the intraoral scan data to 2D image data and/or 3D image data. The image data is then forwarded to the wireless interface 204 which transmits the data to an external device.

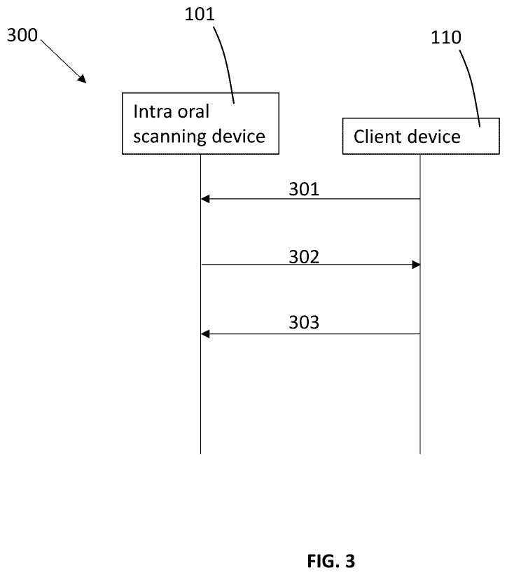

FIG. 3 shows an exemplary sequence diagram 300 between an intraoral scanning device 10 and a client device 110. In an embodiment, the client device 110 may be in the form of a customization device. The intraoral scanning device 10 receives a customization mode request 301 via the wireless interface 204 from the client device 110, the mode request comprising a digital signature and a mode identifier. The digital signature may be a signature according to the Digital Signature Standard or other suitable standards, such as RSA. for digital signatures known in the art. The intraoral scanning device 10 authenticates the mode request by verifying the digital signature. In the illustrated sequence diagram 300, the authentication succeeds, and the processing unit 202 places the intraoral scanning device 10 in the customization mode including sending a customization mode response 302 to the client device via the wireless interface 204. In the customization mode of the intraoral scanning device 10, a firmware part of the memory is write-protected and a customized mode part of the memory is write-enabled.

Upon receipt of the customization mode response 302, the client device 110 sends data 303 to the intraoral scanning device 10 which receives the data and authenticates the received data 303, e.g. by use of digital signature or a session identifier/key as described earlier. If authentication of data 303 succeeds, the processing unit 202 derives intraoral scanning device data (customization data) from the data 303 and stores intraoral scanning device data (customization data) in a customization part of the memory. If authentication of data 303 fails, the processing unit 202 places the intraoral scanning device in default mode.

When the customization data have been transferred, the client device may send a mode exit request and the intraoral scanning device is configured to optionally authenticate the mode exit request and to place the intraoral scanning device in the default mode, optionally if authentication of the mode exit request succeeds.

In another embodiment, the client device may be in the form of a smart phone or a tablet and may comprise software configured to provide the functionality of a customization device.

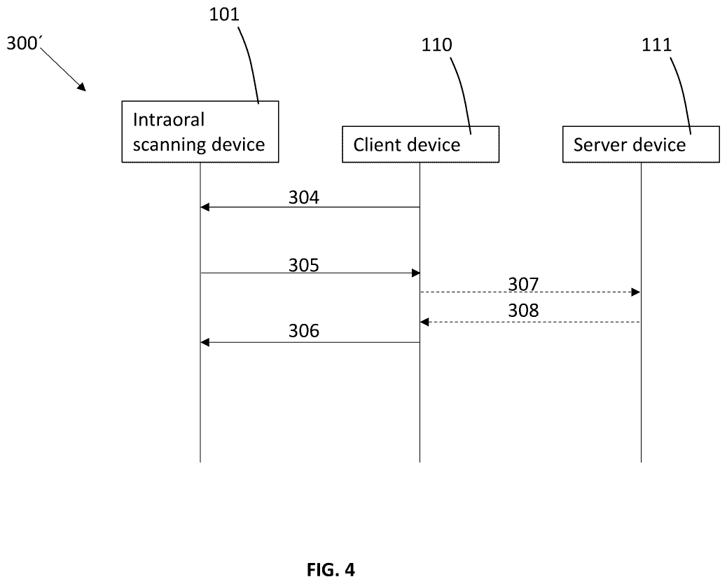

FIG. 4 shows an exemplary sequence diagram 300′ where a client device 110 is used for updating firmware of the intraoral scanning device 10, and a client device 110 in the form of a customization device. The customization device 10 receives a service mode request 304 via the wireless interface 204 from the client device 110. The intraoral scanning device 10 authenticates the service mode request. In the illustrated sequence diagram 300′, the authentication succeeds, and the processing unit 202 places the intraoral scanning device 10 in the service mode including sending a service mode response 305 to the client device via the wireless interface 204. In the service mode of intraoral scanning device 10, the processing unit 202 is allowed to write to a firmware part of the memory.

Upon receipt of the service mode response 305, the client device 110 sends data 306 to the intraoral scanning device 10 which receives the data and authenticates the received data 306, e.g. by use of digital signature or a session identifier/key as described earlier. Before sending data to the intraoral scanning device, the client device 110 may correspond with a server device 111 as illustrated with dotted arrows 307, 308, e.g. in order to determine the data 306 to be sent to the intraoral scanning device 10. If authentication of data 306 succeeds, the processing unit 202 derives intraoral scanning device data (firmware data) from the data 306 and stores intraoral scanning device data (firmware data) in a firmware part of the memory. If authentication of data 306 fails, the processing unit 202 may place the intraoral scanning device in default mode and/or terminate the session.

When the firmware has been transferred, the client device may send a mode exit request and the intraoral scanning device is configured to optionally authenticate the mode exit request and place the intraoral scanning device in the default mode, optionally if authentication of the mode exit request succeeds.

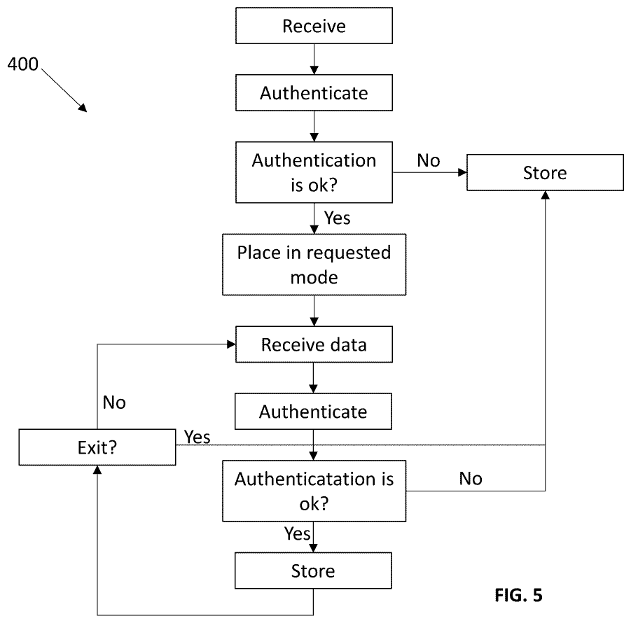

FIG. 5 illustrates an exemplary flowchart of a method 400, e.g. for configuration of a intraoral scanning device 10, comprising a processing unit 202 configured to process intraoral scan data of a patient and provide 2D image data and/or 3D image data, a memory, and a wireless interface 204. The method 400 comprises receiving 401 a mode request via the wireless interface 204 and authenticating 402 the mode request. Authenticating 402 the mode request comprises authenticating the sender of the mode request and verifying integrity of the mode request. If authentication of the mode request succeeds 404, the method proceeds to placing 403 the intraoral scanning device 10 into the requested mode. If authentication of the mode request fails 404, the method optionally proceeds to placing 405 the intraoral scanning device 10 into a default mode. After placing the intraoral scanning device 10 in the requested mode, the method optionally proceeds to receiving 408 data via the wireless interface, authenticating 410 the received data; and storing 412 intraoral device data in a part of the memory corresponding to the requested mode and based on the received data if authentication of the data succeeds. If authenticating 410 the received data fails, the method may proceed to placing 405 the intraoral scanning device in default mode or another mode and/or terminating the session. Upon storing, the method 400 optionally comprises to evaluate 414 whether a mode exit request has been received. If so, the method proceeds to placing 405 the intraoral scanning device in default mode. If not, the method proceeds to receiving 408 data.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/ unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is Intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/o” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. A handheld intraoral scanning device for acquiring intraoral scan data from a three-dimensional dental object during a scanning session, the handheld intraoral scanning device comprising:
a processing unit configured to process intraoral scan data of a patient and provide 3D image data;
a wireless interface configured to transmit the the 3D image data; and
a memory
wherein the processing unit is configured to:
receive a mode request via the wireless interface when no 3D image data is transmitted, wherein the mode request is one or more of a service mode request for a service mode, a customization mode request for customizing a user interface of the handheld intraoral scanning device; an upgrade mode request for upgrading the handheld intraoral scanning device, and a debug mode request, wherein the service mode is firmware part of the memory is writable;
authenticate the mode request to confirm that the mode request is valid for the handheld intraoral scanning device; and
place the handheld intraoral scanning device into the requested mode if authentication of the mode request succeeds.

2. The handheld intraoral scanning device according to claim 1, wherein the processing unit is configured to place the intraoral scanning device into a default mode if authentication of the mode request fails.

3. The handheld intraoral scanning device according to claim 2, wherein the default mode comprises booting the handheld intraoral scanning device and operating the handheld intraoral scanning device according to operating parameters set during booting.

4. The handheld intraoral scanning device according to claim 1, wherein the processing unit is configured to authenticate the mode request by authenticating the sender of the mode request.

5. The handheld intraoral scanning device according to claim 1, wherein the processing unit is configured to authenticate the mode request by verifying integrity of the mode request.

6. The handheld intraoral scanning device according to claim 1, wherein to place the handheld intraoral scanning device into the requested mode if authentication of the mode request succeeds comprises sending a mode response.

7. The handheld intraoral scanning device according to claim 1, wherein the mode request is received in a session and the processing unit is configured to terminate the session if authentication of the mode request fails.

8. A handheld intraoral scanning device according to claim 1, wherein the mode request comprises a signature, and wherein to authenticate the mode request comprises to verify the signature of the mode request.

9. The handheld intraoral scanning device according to claim 1, wherein when the handheld intraoral scanning device is in a service mode, the processing unit is configured to generate a session identifier, to transmit the session identifier via the wireless interface and to store the session identifier in the handheld intraoral scanning device.

10. The handheld intraoral scanning device according to claim 9, wherein when the handheld intraoral scanning device is in a service mode, the processing unit is configured to receive data via the wireless interface, wherein the processing unit is configured to authenticate the received data and store intraoral scanning device data in a part of the memory based on the received data if authentication of the data succeeds.

11. The handheld intraoral scanning device according to claim 10, wherein the data comprises a session identifier, and wherein to authenticate the data comprises comparing the received session identifier with the session identifier stored in the handheld intraoral scanning device.

12. The handheld intraoral scanning device according to claim 10, wherein the data is received in a session and the processing unit is configured to terminate the session if authentication of the received data fails.

13. A method for configuration of a handheld intraoral scanning device comprising a processing unit configured to process intraoral scan data of a patient and provide 3D image data, a memory, and a wireless interface configured to transmit the 3D image data, the method comprising:
receiving a mode request via the wireless interface when no 3D image data is being transmitted, wherein the mode request is one or more of a service mode request for updating firmware data, a customization mode request for customizing a user interface of the handheld intraoral scanning device, an upgrade mode request for upgrading the handheld intraoral scanning device, and a debug mode request, and wherein the service mode is a firmware part of the memory is writable;
authenticating the mode request to confirm that the mode request is valid for the handheld intraoral scanning device; and
placing the handheld intraoral scanning device into the requested mode if authentication of the mode request succeeds.

14. The method according to claim 13, the method comprising placing the handheld intraoral scanning device into a default mode if authentication of the mode request fails.

15. The method according to claim 13, wherein authenticating the mode request comprises authenticating the sender of the mode request.

16. The method according to claim 13, wherein authenticating the mode request comprises verifying integrity of the mode request.

17. The method according to claim 13, wherein when the handheld intraoral scanning device is in a service mode, the method comprises:
receiving data via the wireless interface,
authenticating the received data; and
storing intraoral scanning device data in a part of the memory based on the received data if authentication of the data succeeds.
